(19) United States
(12) Patent Application Publication
(10) Pub. No.: US 2020/0105391 A1
(43) Pub. Date: Apr. 2, 2020

(54) DYNAMIC MANAGEMENT OF TREATMENTS FOR ONE OR MORE CONDITIONS

(71) Applicant: CERNER INNOVATION, INC., Kansas City, KS (US)

(72) Inventors: Shyam Sunder Komirishetty, Austin, TX (US); Vinod Mohanan, Medford, MA (US); Vikrant Malik, Arlington, VA (US); Kristine Marie Nessa, University Heights, OH (US); Brandon M. Smith, Washington, DC (US); Jessica Herstek, Washington, DC (US)

(21) Appl. No.: 16/572,952

(22) Filed: Sep. 17, 2019

Related U.S. Application Data
(60) Provisional application No. 62/739,495, filed on Oct. 1, 2018.

(51) Int. Cl. G16H 20/10; G06F 16/90

(52) U.S. Cl. CPC G16H 20/10; G06F 16/90

(57) ABSTRACT

Computerized systems and methods are provided to intelligently and dynamically manage treatment for one or more conditions for an individual. A manager is programmed to receive an indication to provide one or more active treatments associated with one or more conditions for an individual and then retrieve the one or more active treatments associated with the one or more conditions. The system communicates at least one of the one or more active treatments to a treatment repository. A first criteria is identified, which is applied to one or more potential treatments identified, resulting in the identification of a first subset. Additionally, a second criteria is identified and applied to the first subset, resulting in a second subset comprising potential treatments that satisfy the first and second criteria. The second subset is presented to a user so that the user may make treatment decisions related to the one or more conditions for the individual.

### CLAIM OF PRIORITY

This application, having attorney docket number 27098.296728 and entitled “Dynamic Management of Treatments for One or More Conditions,” claims priority to U.S. Provisional Patent Application No. 62/739,495 filed on Oct. 1, 2018, having attorney docket number 27098.296728, and entitled, “Dynamic Management of Treatments for One or More Conditions,” the entirety of which is incorporated here by reference.

### BACKGROUND

Historically, when a healthcare provider is reviewing treatment options for an individual, they are provided with significant amounts of data regarding the individual's treatment(s) and condition(s) history that can be overwhelming and cumbersome, especially in situations where a healthcare provider needs to make quick treatment decisions for an individual regarding a specific condition. As such, when a healthcare provider must make a treatment analysis and recommendation for an individual, they generally have to review a long list of prior and current treatments for the individual. Often times, the healthcare provider only needs to address one or more specific conditions and therefore, only needs and wants to review treatment(s) related to the one or more specific conditions in order to quickly analyze and make treatment decisions to address the individual's condition(s).

### SUMMARY

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter. The present invention is defined by the claims.

Often times, when a healthcare provider is making treatment determinations for an individual, the healthcare provider primarily relies on the health record of the individual to inform their decisions. Through the utilization of electronic health records, healthcare providers have access to an abundance of data regarding the condition(s) to be treated, treatment options, the individual's health history, and the like. As such, at times, the healthcare provider may be overwhelmed with the amount of information presented or be presented with irrelevant information, especially in circumstances when the healthcare provider needs to make a treatment determination for a specific condition in a short amount of time. Therefore, receiving such an abundance of information which may include each and every treatment, past and present, that an individual has taken, creates the potential for errors in treatment determination. The systems and methods described herein provide customized treatment options by categorizing treatments related to one or more specific conditions being treated and presenting only those treatments to a provider that have satisfied identified criteria. This system minimizes the information presented to the healthcare provider, thereby allowing for the healthcare provider to make targeted treatment decisions based on the condition at hand.

Embodiments of the present invention generally relate to computerized systems and methods that facilitate the dynamic management of treatments for one or more conditions. This reduces information overload to healthcare providers and decreases inefficiencies in the management of treatments for one or more conditions. In accordance with the technology described herein, the system comprises at least one computer server (“server”) and at least one manager. The system is programmed to receive an indication to provide one or more active treatments associated with one or more conditions for an individual and then retrieve the one or more active treatments associated with the one or more conditions. The system also retrieves a unique identifier for each of the one or more active treatments and communicates at least one of the one or more active treatments to a treatment repository. The system identifies a first criteria and one or more potential treatments, and applies the first criteria to the one or more potential treatments identified, resulting in the identification of a first subset that comprises the one or more potential treatments that satisfy the first criteria. The system also identifies a second criteria and applies the second criteria to the first subset to identify a second subset of potential treatments that satisfy both the first criteria and the second criteria. The second subset is provided, by the system, to a user such as a healthcare provider. The system may additionally store the first and second subsets for future use. The system further may apply additional criteria such a third criteria, resulting in additional subsets, which may also be stored for future use.

### BRIEF DESCRIPTION OF THE SEVERAL VIEWS OF THE DRAWINGS

The present invention is described in detail below with reference to the attached drawing figures, wherein:

FIG. 1 is a block diagram of an exemplary system architecture in which embodiments of the invention may be employed;

FIG. 2 is an exemplary system architecture suitable to implement embodiments of the present invention;

FIG. 3 is an exemplary guided user interface illustrating data associated with an RXCUI code searched;

FIG. 4 is a screenshot of an exemplary health care record for an individual; and

FIG. 5 is a flow diagram showing an exemplary method for managing treatments for one or more conditions.

### DETAILED DESCRIPTION

The subject matter of the present invention is described with specificity herein to meet statutory requirements. However, the description itself is not intended to limit the scope of this patent. Rather, the inventors have contemplated that the claimed subject matter might also be embodied in other ways, to include different steps or combinations of steps similar to the ones described in this document, in conjunction with other present or future technologies. Moreover, although the terms “step” and/or “block” may be used herein to connote different components of methods employed, the terms should not be interpreted as implying any particular order among or between various steps herein disclosed unless and except when the order of individual steps is explicitly described.

Traditional health care management systems present healthcare providers with an exhaustive list of treatments for an individual when the healthcare provider accesses an individual's health record. For example, if a healthcare provider is treating an individual for diabetes, when the healthcare provider pulls up the individuals electronic health record on a computer, the healthcare provider is presented with an exhaustive list of all medications for the individual that is not limited to diabetes or, perhaps, even current medications. As such, the healthcare provider is given significantly more information than is needed and the healthcare provider must manage all the information provided in order to make determinations as to what treatment the individual should be given for diabetes. For consistency purposes, diabetes is used as the exemplary condition throughout this disclosure. However, the system and methods disclosed herein may be applicable to a variety of other conditions, including but not limited to asthma, heart disease, and arthritis.

Depending on the situation, a healthcare provider may not need or want to review each treatment for any and all conditions associated with the individual. Instead, the healthcare provider prefers to review only those treatments associated with the one or more conditions. Therefore, the healthcare provider treating diabetes would prefer to review only the treatments associated with diabetes management for the individual and then make quick clinical decisions based on the targeted and narrow treatment information provided. This overload of information is inefficient as the healthcare provider has to review all treatments prior to taking action. As such, there is a need for treatment categorization to improve efficiency of health care management. A system that receives/retrieves data such as lab results and active treatments related to the one or more conditions and utilizes this information to provide potential treatments that meet identified criteria provides a more focused approach to allow a healthcare provider to make quick decisions related to treatment of the one or more conditions. This leads to reduced costs as healthcare providers can make more focused treatment decisions in faster time, which may also result in additional time for a healthcare provider to treat more individuals within a work day.

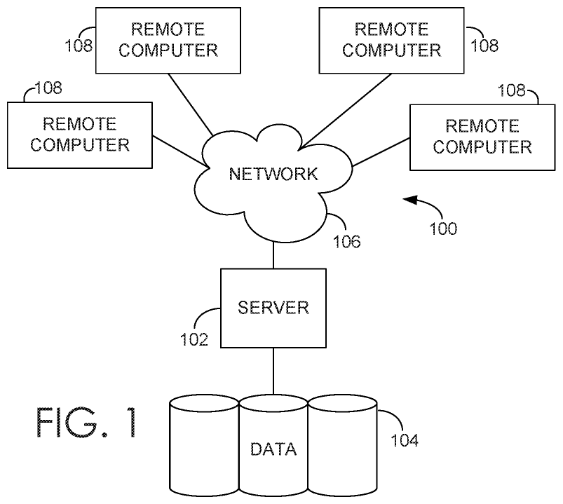

Beginning with FIG. 1, an exemplary computing environment suitable for use in implementing embodiments of the present invention is shown. FIG. 1 is an exemplary computing environment (e.g., health-information computing-system environment) with which embodiments of the present invention may be implemented. The computing environment is illustrated and designated generally as reference numeral 100. The computing environment 100 is merely an example of one suitable computing environment and is not intended to suggest any limitation as to the scope of use or functionality of the invention. Neither should the computing environment 100 be interpreted as having any dependency or requirement relating to any single component or combination of components illustrated therein. It will be appreciated by those having ordinary skill in the art that the connections illustrated in FIG. 1 are also exemplary as other methods, hardware, software, and devices for establishing a communications link between the components, devices, systems, and entities, as shown in FIG. 1, may be utilized in the implementation of the present invention. Although the connections are depicted using one or more solid lines, it will be understood by those having ordinary skill in the art that the exemplary connections of FIG. 1 may be hardwired or wireless, and may use intermediary components that have been omitted or not included in FIG. 1 for simplicity's sake. As such, the absence of components from FIG. 1 should not be interpreted as limiting the present invention to exclude additional components and combination(s) of components. Moreover, though devices and components are represented in FIG. 1 as singular devices and components, it will be appreciated that some embodiments may include a plurality of the devices and components such that FIG. 1 should not be considered as limiting the number of a device or component.

The present technology might be operational with numerous other special-purpose computing system environments or configurations. Examples of well-known computing systems, environments, and/or configurations that might be suitable for use with the present invention include personal computers, server computers, hand-held or laptop devices, multiprocessor systems, microprocessor-based systems, set top boxes, programmable consumer electronics, network PCs, minicomputers, mainframe computers, distributed computing environments that include any of the above-mentioned systems or devices, and the like.

The present invention may be operational and/or implemented across computing system environments such as a distributed or wireless “cloud” system. Cloud-based computing systems include a model of networked enterprise storage where data is stored in virtualized storage pools. The cloud-based networked enterprise storage may be public, private, or hosted by a third party, in embodiments. In some embodiments, computer programs or software (e.g., applications) are stored in the cloud and executed in the cloud. Generally, computing devices may access the cloud over a wireless network and any information stored in the cloud or computer programs run from the cloud. Accordingly, a cloud-based computing system may be distributed across multiple physical locations.

The present technology might be described in the context of computer-executable instructions, such as program modules, being executed by a computer. Exemplary program modules comprise routines, programs, objects, components, and data structures that perform particular tasks or implement particular abstract data types. The present invention might be practiced in distributed computing environments where tasks are performed by remote processing devices that are linked through a communications network. In a distributed computing environment, program modules might be located in association with local and/or remote computer storage media (e.g., memory storage devices).

With continued reference to FIG. 1, the computing environment 100 comprises a computing device in the form of a control server 102. Exemplary components of the control server 102 comprise a processing unit, internal system memory, and a suitable system bus for coupling various system components, including data store 104, with the control server 102. The system bus might be any of several types of bus structures, including a memory bus or memory controller, a peripheral bus, and a local bus, using any of a variety of bus architectures. Exemplary architectures comprise Industry Standard Architecture (ISA) bus, Micro Channel Architecture (MCA) bus, Enhanced ISA (EISA) bus, Video Electronic Standards Association (VESA) local bus, and Peripheral Component Interconnect (PCI) bus, also known as Mezzanine bus.

The control server 102 typically includes therein, or has access to, a variety of non-transitory computer-readable media. Computer-readable media can be any available media that might be accessed by control server 102, and includes volatile and nonvolatile media, as well as, removable and nonremovable media. By way of example, and not limitation, computer-readable media may comprise computer storage media and communication media. Computer storage media includes volatile and nonvolatile, removable and non-removable media implemented in any method or technology for storage of information such as computer-readable instructions, data structures, program modules or other data. Computer storage media includes, but is not limited to, RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other medium which can be used to store the desired information and which can be accessed by control server 102. Computer-readable media does not include signals per se.

Communication media typically embodies computer-readable instructions, data structures, program modules or other data in a modulated data signal such as a carrier wave or other transport mechanism and includes any information delivery media. The term “modulated data signal” means a signal that has one or more of its characteristics set or changed in such a manner as to encode information in the signal. By way of example, and not limitation, communication media includes wired media such as a wired network or direct-wired connection, and wireless media such as acoustic, RF, infrared and other wireless media. Combinations of any of the above should also be included within the scope of computer-readable media.

The control server 102 might operate in a computer network 106 using logical connections to one or more remote computers 108. Remote computers 108 might be located at a variety of locations including operating systems, device drivers and medical information workflows. The remote computers might also be physically located in traditional and nontraditional medical care environments so that the entire medical community might be capable of integration on the network. The remote computers might be personal computers, servers, routers, network PCs, peer devices, other common network nodes, or the like and might comprise some or all of the elements described above in relation to the control server. The devices can be personal digital assistants or other like devices. Further, remote computers may be located in a variety of locations including in a medical or research environment, including clinical laboratories (e.g., molecular diagnostic laboratories), hospitals and other inpatient settings, veterinary environments, ambulatory settings, medical billing and financial offices, hospital administration settings, home healthcare environments, and clinicians' offices. Healthcare providers may comprise a treating physician or physicians; specialists such as surgeons, radiologists, cardiologists, and oncologists; emergency medical technicians; physicians' assistants; nurse practitioners; nurses; nurses' aides; pharmacists; dieticians; microbiologists; laboratory experts; laboratory technologists; genetic counselors; researchers; veterinarians; students; and the like. The remote computers 108 might also be physically located in nontraditional medical care environments so that the entire medical community might be capable of integration on the network. The remote computers 108 might be personal computers, servers, routers, network PCs, peer devices, other common network nodes, or the like and might comprise some or all of the elements described above in relation to the control server 102. The devices can be personal digital assistants or other like devices.

Computer networks 106 comprise local area networks (LANs) and/or wide area networks (WANs). Such networking environments are commonplace in offices, enterprise-wide computer networks, intranets, and the Internet. When utilized in a WAN networking environment, the control server 102 might comprise a modem or other means for establishing communications over the WAN, such as the Internet. In a networking environment, program modules or portions thereof might be stored in association with the control server 102, the data store 104, or any of the remote computers 108. For example, various application programs may reside on the memory associated with any one or more of the remote computers 108. It will be appreciated by those of ordinary skill in the art that the network connections shown are exemplary and other means of establishing a communications link between the computers (e.g., control server 102 and remote computers 108) might be utilized.

In operation, an organization might enter commands and information into the control server 102 or convey the commands and information to the control server 102 via one or more of the remote computers 108 through input devices, such as a keyboard, a microphone (e.g., voice inputs), a touch screen, a pointing device (commonly referred to as a mouse), a trackball, or a touch pad. Other input devices comprise satellite dishes, scanners, or the like. Commands and information might also be sent directly from a remote medical device to the control server 102. In addition to a monitor, the control server 102 and/or remote computers 108 might comprise other peripheral output devices, such as speakers and a printer.

Although many other internal components of the control server 102 and the remote computers 108 are not shown, such components and their interconnection are well known. Accordingly, additional details concerning the internal construction of the control server 102 and the remote computers 108 are not further disclosed herein.

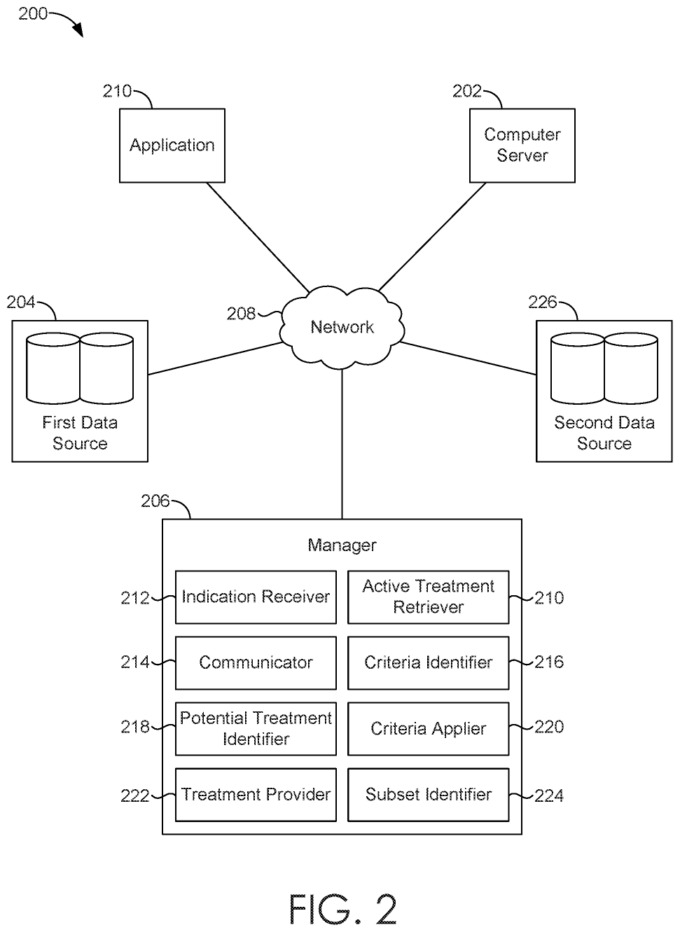

Turning now to FIG. 2, an exemplary computing system 200 is depicted. The computing system 200 is merely an example of one suitable computing system and is not intended to suggest any limitation as to the scope of use or functionality of embodiments of the present invention. Neither should the computing system 200 be interpreted as having any dependency or requirement related to any single component or combination of components illustrated herein.

In the embodiment shown in FIG. 2, the system 200 includes at least one computer server 202, at least one application 210, a manager 206, a first data source 204, a second data source 226, and a network 208. While FIG. 2 illustrates only one computer server 202, it is contemplated that the system 200 may comprise any number of servers 202. Additionally, the application 210 may be located on any user device.

A first data source 204 may be any data source, including treatment repositories that the system 200 communicates with to manage treatment for one or more conditions. In one embodiment, the first data source 204 is a treatment repository that may be a public source for use. For example, an exemplary data source may be RxNav®, a treatment repository run by the United States Library of Medicine. RxNav® is a browser for several treatment (e.g. drug) information sources that finds drugs in RxNorm®, a normalized naming system for generic and brand-name drugs. RxNav® provides a tool for supporting interoperation between drug terminologies and pharmacy knowledge base systems. Hospitals, pharmacies, and other organizations use computer systems to record and process drug information. However, since these systems may use different sets of drug names, it can be difficult for one system to communicate with another. To resolve this issue, RxNorm® provides normalized names and unique identifiers for medicines and drugs. A user may access the RxNav® website (http://mor.nlm.nih.gov/RxNav/#) and enter in a name of a treatment (e.g. ibuprofen) or a unique identifier associated with the treatment. The unique identifier associated with each treatment is called an RxNorm® Concept Unique Identifier or RXCUI code.

In other embodiments, the first data source 204 is a third party treatment repository or the system may have its own treatment repository. In this case, the first data source 204 may function similar to the RxNav® treatment repository and may provide a browser for searching drug information. A user may enter in a drug name or unique identifier similar to a RXCUI code. Once entered, the first data source 204 may pull up a variety of information regarding the treatment, including but not limited to ingredients, brand names, clinical dosages, and clinical drug components.

As depicted, the system 200 is comprised of one manager 206, but it is contemplated the system 200 may include more than one manager 206. It will be appreciated that some or all of the subcomponents of the manager 206 may be accessed via the network 208 and may reside on one or more devices. Additionally, the manager 206 may also be integrated into the application 210. Further, in some embodiments, one or more of the illustrated components may be implemented as a stand-alone application. The components described are exemplary in nature and in number and should not be construed as limiting. Any number of components may be employed to achieve the desired functionality within the scope of the embodiments hereof.

Generally, the manager 206 is configured to dynamically manage the treatments for one or more conditions. In embodiments, the manager 206 may be configured to access the at least one server 202 at any time based on receiving an indication to provide one or more active treatments to a user or healthcare provider for one or more conditions associated with that individual. As shown, the manager 206 is comprised of several components including: an active treatment retriever 210, an indication receiver 212, a communicator 214, a criteria identifier 216, a potential treatment identifier 218, a criteria applier 220, a treatment provider 222, and a subset identifier 224. In other embodiments, the manager 206 may include any number of components necessary for the dynamic management of one or more treatments for one or more conditions.

The indication receiver 212 within the manager 206 is configured to receive an indication to provide one or more active treatments associated with one or more conditions for an individual. The indication receiver 212 may receive the indication to provide the one or more active treatments from a healthcare provider who is reviewing the individual's health care profile in order to determine proper treatment for one or more conditions associated with the individual. In this embodiment, the indication receiver 212 may be requested to provide one or more active treatments which are the current treatments utilized by the individual for the one or more conditions associated with the individual. In other embodiments, it is contemplated that the indication receiver 212 may also receive requests to provide both past and current treatments associated with the one or more conditions. For example, the system may receive an indication, from a healthcare provider managing diabetes treatment for an individual to provide one or more active treatments associated with diabetes management for that individual. The indication received from the healthcare provider is specific in an effort to avoid presenting the healthcare provider with an overload of treatments for any and all conditions associated with the individual's health. Additionally, in some instances, the indication receiver 212 may receive an indication to provide one or more treatments associated with an additional condition. For example, if the healthcare provider is managing treatment of an individual who suffers from asthma in addition to diabetes, the system may receive an indication to provide one or more active treatments associated with asthma in addition to those treatments associated with diabetes.

After the indication receiver 212 receives an indication to provide one or more active treatments associated with the one or more conditions (e.g., one or more conditions specified by a provider), an active treatment retriever 210 is configured to retrieve the one or more active treatments associated with the one or more conditions for the individual. When the active treatment retriever 210 retrieves one or more active treatments associated with the one or more conditions for the individual, it also retrieves a unique identifier for each of the one or more active treatments. As previously mentioned, a unique identifier may be an RXCUI code or any other unique identifier that is assigned to each treatment. The unique identifier retrieved is important as it allows for interoperability and clear communication between various systems regarding different treatments. For example, different health care systems may utilize different brand and generic treatments to treat the same condition. As such, the assigned unique identifier or RXCUI code ties together treatments which have common active ingredients. This is useful for healthcare providers so that they are able to view a full list of treatments that fall within one RXCUI code or unique identifier.

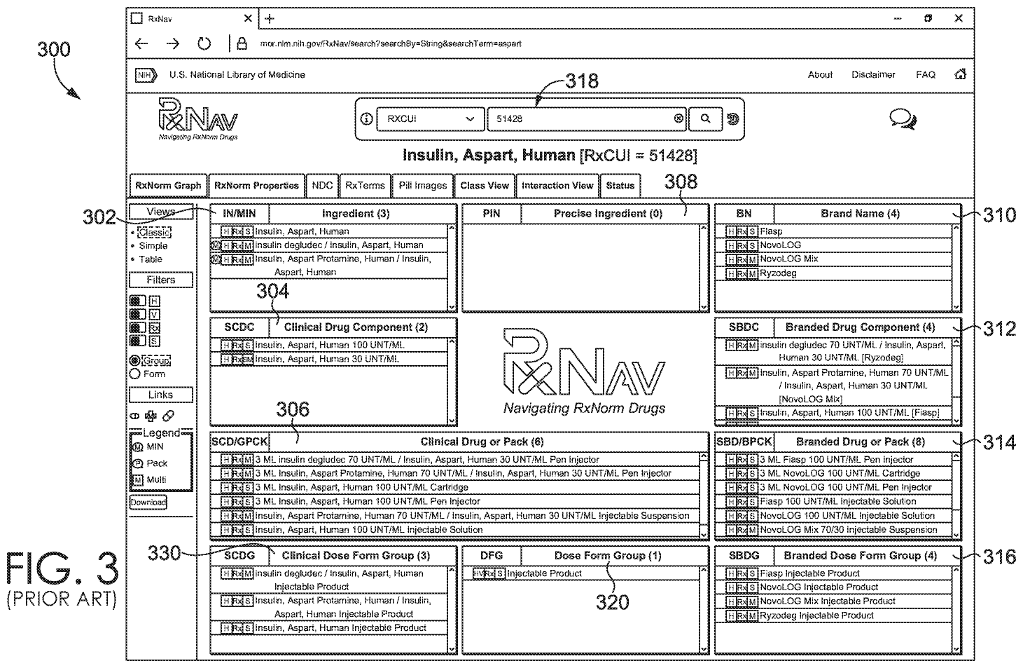

The communicator 214 communicates at least one of the one or more active treatments to a first data source 204. As mentioned, the first data source 204 may be any source that stores information related to treatments for conditions. In one embodiment, the first data source 204 may be a treatment repository that is run by the system, a third party, or a repository such as RxNav® (provided by the U.S. National Library of Medicine). As will be described further in FIG. 3, communicating at least one of the one or more active treatments to the RxNav® repository may comprise entering either the treatment name (e.g. ibuprofen) or an RXCUI code (e.g. 84518) into the search field of the RxNav® web browser.

The system identifies a first criteria via the criteria identifier 216 from the first data source 204. In this embodiment, a first criteria may be the active ingredient of the treatment. Once the system has communicated the one or more active treatments to the treatment repository via the communicator 214, RxNav® provides a list of ingredients for the active treatment. While the first criteria in the exemplary embodiment is an active ingredient, the first criteria may be any variety of other characteristics attributed to the treatment communicated. For example, the first criteria may be a dosage, delivery mechanism, or brand name.

A potential treatment identifier 218 identifies one or more potential treatments associated with one or more conditions. For example, for diabetes, the potential treatment identifier 218 may identify all potential treatments for diabetes. Once the potential treatments have been identified, the criteria applier 220 applies the first criteria to the potential treatments identified. For diabetes, the first criteria identified may be Insulin, Aspart, Human with RXCUI code 51428. The criteria applier 220 will take the one or more potential treatments identified for diabetes and determine which potential treatments comprise of the first criteria, Insulin, Aspart, Human. Depending on the number of potential treatments that were identified by the potential treatment identifier 218, there may be zero or more potential treatments which comprise the first criteria. Once the first criteria (Insulin, Aspart, Human) is applied to the potential treatments identified, a subset identifier 224 identifies a first subset, which comprises one or more potential treatments that satisfy the first criteria. By way of example, if the potential treatment identifier 218 had identified 10 potential diabetes treatments, once the first criteria is applied, the subset identifier 224 may only identify three potential treatments, such as Fiasp®, NovoLog®, and Ryzodeg®, all of which comprise the active ingredient Insulin, Aspart, Human.

Additionally, the criteria identifier 216 may identify a second criteria to be applied to the first subset. The second criteria to be applied may be any further identifying criteria such as delivery mechanism, dosage, brand/generic drug, or any other characteristic that defines treatments. Further, the second criteria may be determined by the healthcare provider or by the system. In the present embodiment, the second identifier may be a delivery mechanism for the treatment such as oral or intravenous administration. As such, the criteria applier 220 will apply the second criteria, or delivery mechanism to the first subset of treatments. This is important as some treatments are manufactured for oral use only, intravenous use only, or both. For example, if the delivery mechanism is intravenous administration, the criteria applier will take the three potential treatments which satisfied the first criteria (from the above example), Fiasp®, NovoLog®, and Ryzodeg® and apply the second criteria, intravenous administration. When the second criteria is applied, all or less than all of the potential treatments may have intravenous administration options. Therefore, for exemplary purposes, if Fiasp® and NovoLog® could be administered intravenously, but Ryzodeg® can only be administered orally, then only Fiasp® and NovoLog® would meet the second criteria (this determination is merely exemplary and, as such, the delivery mechanisms of these example treatments may differ in actual clinical use). Based on this, the subset identifier 224 would identify a second subset that includes Fiasp® and NovoLog®. This second subset comprises the one or more potential treatments that satisfied both the first criteria, active ingredient Insulin, Aspart, Human and the second criteria, intravenous administration. Following this, the treatment provider 222 may provide the second subset to a user, such as a healthcare provider, for determination of which potential treatments to administer to the individual. It is also contemplated that in other embodiments, the system, utilizing treatment provider 222, may also provide both the first subset and second subset of potential treatments.

In further embodiments, the criteria identifier 216 identifies a third criteria in addition to the first and second criteria. In the present example, the criteria applier 220 may apply a third criteria such as dosage amount in addition to the first and second criteria. The criteria identifier 216 may identify a dosage amount desired for the individual and condition and the criteria applier 220 may apply the third criteria comprising the dosage amount to the second subset prior to providing the potential treatments to the healthcare provider. The additional criteria would enable further narrowing of the number of potential treatments provided to the healthcare provider for the one or more conditions associated with the individual, thereby decreasing the time the healthcare provider will spend reviewing the potential treatments, and making the process more efficient and targeted for each individual condition being treated.

Once the third criteria is applied, a third subset comprising the one or more potential treatments that satisfy the first, second, and third criteria will be identified by the subset identifier 224. The treatment provider 222 provides the third subset (and potentially the first and second or any combination thereof) to the user for treatment decisions. Continuing with the diabetes example, a third criteria, for exemplary purposes, may be a 100 unit/mL injectable solution. When this third criteria is applied by the criteria applier 220 to the second subset comprising Fiasp® and NovoLog® treatments, only the treatments which may be dosed at 100 unit/mL injectable solution will satisfy the third criteria and become a part of the third subset. Therefore, if Fiasp® is available via injectable solution, but only in 30 Units/mL, Fiasp® would not satisfy the third criteria and would be eliminated as a potential treatment to be presented to the user by the treatment provider 222.

Further, it is contemplated that in some embodiments, the potential treatments identified by the system, including the first subset and second subset may be stored for future use at a data source. For example, the potential treatments identified by the system may be stored at a single repository such as the first data source 204, at a second data source 226, or may be stored at the first data source 204 and potentially backed up at a second data source 226. The first data source 204 and second data source 226 may be located in the cloud or on a remote server. Alternatively, either or both data sources could be located (or backed up) locally.

The storage of the identified subsets may be especially beneficial so that when a first data source 204 may be turned off or “go down” due to updates or problems such as power outages, the system will still be able to function and present potential treatments to users for the management of one or more conditions for an individual. Additionally, if the subsets identified are stored, the system could utilize the stored information for management of additional individuals with similar conditions. For example, if a healthcare provider is managing a second individual's diabetes and the individual presents with the same type of diabetes (e.g. Type 2) and similar other factors, the system may pull the previously identified subsets and present the potential treatments in those subsets to the healthcare provider. This would further improve the efficiency for treatment management as the system would be able to provide the potential treatment options to the healthcare provider almost instantly, allowing the healthcare provider to make treatment determinations sooner, which may also result in the individual receiving care and a quicker improved health status for the individual in some circumstances.

Additionally, the system, at predetermined intervals, may communicate, via the communicator 214, with the first data source 204 or treatment repository (e.g. RxNav®) to dynamically update the potential treatments that satisfy each criteria. This is particularly important as the treatments available for managing conditions are constantly being updated and there may be changes to existing treatments (e.g. dosage, delivery mechanism, composition, etc.) or new treatments that a healthcare provider should be viewing when making treatment decisions for the management of the condition associated with the individual. The communication and updating of the potential treatments that have the first, second, and potentially additional criteria applied to is critical so that healthcare providers maintain and satisfy meeting standard levels of care and make decisions on treatments based on the most updated and relevant treatment information available. The filtering of the potential treatments by the system simplifies the process for a healthcare provider, allowing the focus of their decision to be on potential treatments meeting criteria that are relevant or necessary for a specific condition or individual based on the individual's overall health record and status.

Next, FIG. 3 illustrates an exemplary guided user interface (“interface”) 300 illustrating data associated with an RXCUI code searched. The interface 300 may be from a first data source 204, such as a treatment repository, that the system may communicate with to identify the first criteria. In this case, the first data source 204, is the RxNav® treatment repository. However, the treatment repository is not limited to RxNav®. As such, the first data source 204 may be any data source comprising data that is relevant to the management of treatments for one or more conditions. As previously mentioned, RxNav® is a browser for several drug information sources and provides data for clinical drugs, both brand-name and generic, regarding active ingredients, and drug components. As shown in FIG. 3, the one or more potential treatments have been communicated to the RxNav® treatment repository. The potential treatments may be communicated by, for example, the communicator 214. In the interface 300, the RXCUI code 51428, has been entered into the search field 318. Once searched, RxNav® returns the ingredient—Insulin, Aspart, Human. RxNav® also provides a variety of information including the ingredients 302, clinical drug component(s) 304, clinical drug/pack(s) 306, clinical dose form group 330, dose form group(s) 320, brand names 310, precise ingredient(s) 308, brand drug component(s) 312, branded drug or pack(s) 314, and branded dose form group(s) 316.

From the information presented on interface 300, a first criteria is identified. The first criteria may be identified by, for example, the criteria identifier 216, which may receive instructions regarding the first criteria to be identified from the healthcare provider or by the system. In the example presented, the first criteria is the active ingredient. Therefore, the criteria identifier 216 would identify Insulin, Aspart, Human to be applied to the one or more potential treatments identified by the potential treatment identifier 218. While the active ingredient is the first criteria in the embodiment shown, it is contemplated that the first criteria might be any of the criteria shown on the RxNav® screen such as clinical drug component 304 or brand name 310. Also, in addition to searching the RxNav® 300 treatment repository by RXCUI code, RxNav® can also be searched by ingredient. For example, “aspart” may be entered instead of RXCUI 51428 which would present the same information.

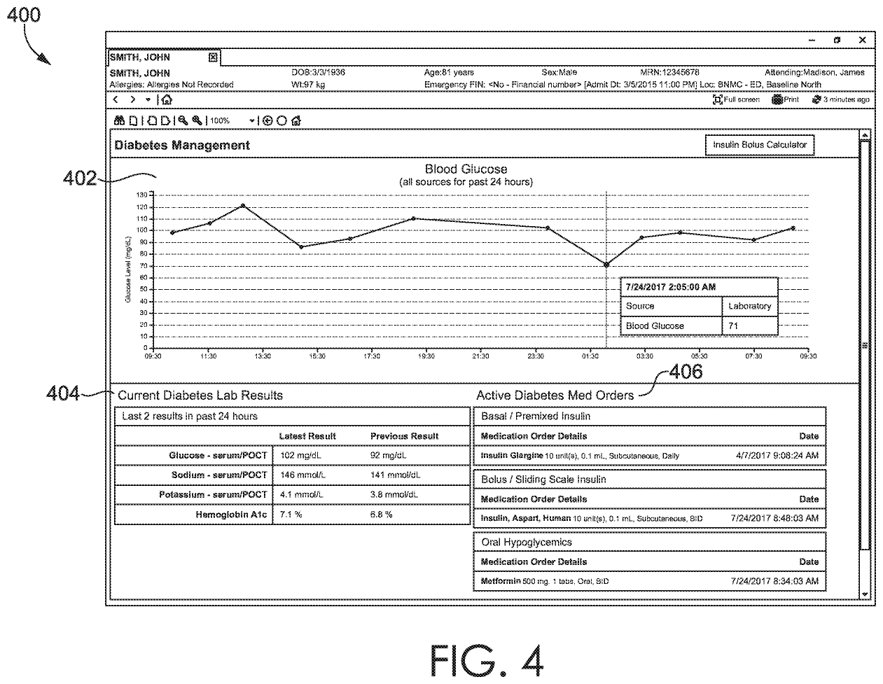

Continuing to FIG. 4, an exemplary screenshot of information from an individual's electronic health record is presented. The image 400 presents multiple pieces of clinical information to a healthcare provider on one screen to make treatment decisions more efficient and easier for the healthcare provider. As shown, the image 400 presents a graph 402 indicating the blood glucose levels for individual John Smith over the last 24 hours. It also presents the Current Diabetes Lab Results 404 (two in the last 24 hours) for glucose, sodium, potassium, and hemoglobin. Additionally, Active Diabetes Med. Orders 406 are presented to the user which show the medication order details and date information. This screen presents the relevant information for treatment decision making in one interface for the healthcare provider to utilize. Based on the potential treatments provided to the healthcare provider by the treatment provider 222, the healthcare provider can analyze the potential treatments along with the data seen in FIG. 4 to make a treatment determination.

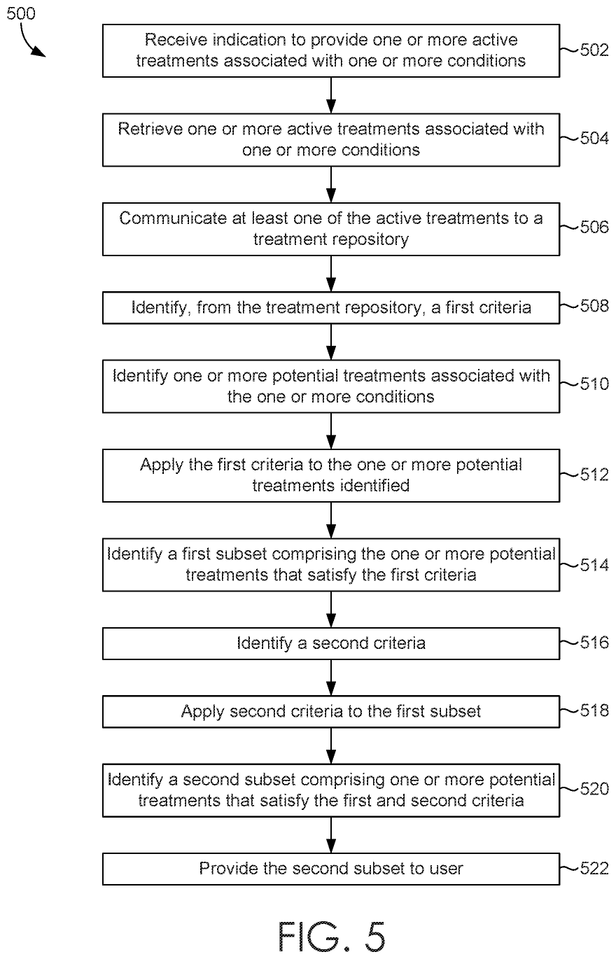

Turning next to FIG. 5, a flow diagram is depicted of an exemplary method 500 for dynamically managing treatments for one or more conditions in an effective and efficient manner so that a healthcare provider, such a physician, can make quick and accurate treatment decisions for an individual. The method 500 may be implemented by the computing system architect 200 described with respect to FIG. 2.

At step 502, the indication receiver 212 receives an indication to provide one or more active treatments associated with one or more conditions for an individual. As previously mentioned, traditionally, when a healthcare provider is reviewing treatments for an individual, they are provided with an exhaustive list of all the treatments for an individual for various different conditions, which is cumbersome and slows down the treatment process. As such, the indication receiver 212 is configured to receive indications to provide one or more active treatments for one or more conditions rather than an exhaustive list of all treatments for an individual.

Next, at step 504, the active treatment retriever 210 will retrieve the one or more active treatments that an indication was received for. When the active treatment retriever 210 retrieves the one or more active treatments, each active treatment retrieved comprises a unique identifier such as an RXCUI code corresponding to the treatment.

Once the system retrieves the one or more treatments with the RXCUI codes, it communicates with a data source, such as a treatment repository at step 506. The communicator 214 communicates at least one of the active treatments to a treatment repository such as RxNav®. As shown in FIG. 3, the RXCUI code is entered into RxNav® in order to obtain a variety of information related to the treatment. Based on the information retrieved, a first criteria is identified by the criteria identifier 216 at step 508.

Next, one or more potential treatments associated with the one or more conditions are identified by potential treatment identifier 218 at step 510. As such, the potential treatment identifier 218 may identify both treatments the individual has previously used and new treatments. The only limitation in place is to identify treatments that are associated with one or more conditions. As one can imagine, the number of potential treatments identified by the potential treatment identifier 218 may be large for diabetes as it is a common condition that affects a large population and the management of the condition varies greatly. Therefore, to narrow the potential treatment options to either be more manageable or focused on the specific issues to be managed with relation to the individual's diabetes, the first criteria or active ingredient, is applied to the potential treatments identified at step 512. As previously mentioned, the first criteria is not limited to the active ingredient for the one or more active treatments communicated to the data source or treatment repository. The first criteria may be any characteristic that is relevant to the treatment determination regarding an individual's condition and may be dictated by the system in some embodiments or by the user in other embodiments.

Once the first criteria is applied by the criteria applier 220 to the potential treatments identified by the potential treatment identifier 218, the subset identifier 224 identifies a first subset that comprises the potential treatments that satisfy the first criteria (e.g. active ingredient) at step 514. Depending on the condition being treated, the number of potential treatments that satisfy the first criteria and are found in the first subset will vary. In the present example for diabetes treatment, if the first criteria is active ingredient and the identified active ingredient is insulin, it is likely the first subset will comprise a number of different potential treatments since the lack of insulin is a critical issue diabetes. By contrast, if the active ingredient identified as the first criteria is some other ingredient, such as Aspart Protoamine, the number of potential treatments that satisfy the first criteria may be fewer.

Next, the criteria identifier 220 is configured to identify a second criteria at step 516. In the present example, the second criteria is the delivery mechanism, but as discussed, the criteria may be any criteria which allows for the narrowing of potential treatments based on relevant factors. Once the second criteria is identified by the criteria identifier 216, the criteria applier 220 will apply the second criteria to the first subset at step 518 in the same way that the criteria applier 220 applied the first criteria to the potential treatments.

At step 520, the subset identifier will identify a second subset that is comprised of one or more potential treatments that satisfy both the first and second criteria. The number of potential treatments in the second subset will vary based on the first criteria, second criteria, condition, and the number of potential treatments identified at step 510. After the second subset is identified at step 520, the treatment provider 222 will provide the second subset to a user (e.g. healthcare provider) at step 522 so that the user can utilize the information in making determinations about the treatment of an individual for the identified condition.

The method 500 may further include (not shown) the additional steps of identifying additional criteria to further narrow the potential treatment options. As such, the criteria identifier 216 may identify additional criteria such as dosage or branded/generic treatments. Based on the additional criteria, the subset identifier 224 will identify subsequent subset of potential treatments that satisfy all the criteria identified and applied. Once all the potential criteria have been identified and applied, the treatment provider 222 will then provide the potential treatments found in the final subset (e.g. third subset, fourth subset, etc.) to the healthcare provider so that the healthcare provider can make treatment decisions for managing the condition for the individual. The number of criteria identified and applied may vary based on the condition, number, and type of potential treatment options identified, and the user's preferences. Further, in some embodiments, it is possible that only a first criteria may be applied.

Additionally, the method 500 may also include the step (not shown) of storing the final subset or all subsets at a second data source 226 for future use. This step will be particularly beneficial in circumstances when an external first data source 204, such as RxNav®, may be temporarily inaccessible. In such cases, the storing of the second (or final if more than two criteria are applied) or all subsets will allow the system to continue to function regardless of the status of the first data source 204. It is contemplated that the storage of the subsets may be local or remote. Additionally, storage of this information may also lead to further efficiencies by providing for repetitive use of the subsets identified for additional individuals who have the same conditions as conditions already identified.

The present invention has been described in relation to particular embodiments, which are intended in all respects to be illustrative rather than restrictive. Further, the present invention is not limited to these embodiments, but variations and modifications may be made without departing from the scope of the present invention.

What is claimed is:

1. A system for dynamically managing treatments for one or more conditions, the system comprising:
receiving an indication to provide one or more active treatments associated with one or more conditions for an individual;
retrieving the one or more active treatments associated with the one or more conditions for the individual, wherein the one or more active treatments comprises a unique identifier;
communicating at least one of the one or more active treatments to a treatment repository;
identifying, from the treatment repository, a first criteria;
identifying one or more potential treatments associated with the one or more conditions;
applying the first criteria to the one or more potential treatments associated with the one or more conditions;
identifying a first subset, wherein the first subset comprises the one or more potential treatments that satisfy the first criteria;
identifying a second criteria;
applying the second criteria to the first subset;
identifying a second subset, wherein the second subset comprises the one or more potential treatments that satisfied both the first and second criteria; and
providing the second subset to a user.

2. The system of claim 1, wherein the unique identifier is an assigned RxNorm® concept unique identifier code (RXCUI).

3. The system of claim 2, wherein each active treatment is assigned a unique RXCUI code.

4. The system of claim 1, wherein the first criteria comprises an active ingredient.

5. The system of claim 1, wherein the second criteria comprises a delivery mechanism for delivering the one or more potential treatments to the individual.

6. The system of claim 5, wherein the delivery mechanism comprises at least one of oral delivery or intravenous delivery.

7. The system of claim 1, wherein the system identifies a third criteria comprising a dosage of the one or more potential treatments.

8. The system of claim 1, wherein the first subset is stored for future use.

9. The system of claim 1, wherein the second subset is stored for future use.

10. The system of claim 1, wherein the system, at predetermined intervals, communicates with the treatment repository to dynamically update the potential treatments that meet the first and second criteria.

11. The system of claim 1, wherein the treatment repository is RxNav®, a web application program interface that provides a list of ingredients for each active treatment communicated.

12. The system of claim 1, wherein the one or more conditions is diabetes.

13. A method for dynamically managing treatments for one or more conditions, the method comprising:
receiving an indication to provide one or more active treatments associated with one or more conditions for an individual;
retrieving the one or more active treatments associated with the one or more conditions for the individual, wherein the one or more active treatments comprises a unique identifier;
communicating at least one of the one or more active treatments to a treatment repository;
identifying, from the treatment repository, a first criteria;
identifying one or more potential treatments associated with the one or more conditions;
applying the first criteria to the one or more potential treatments associated with the one or more conditions;
identifying a first subset, wherein the first subset comprises the one or more potential treatments that satisfy the first criteria;
identifying a second criteria;
applying the second criteria to the first subset;
identifying a second subset, wherein the second subset comprises the one or more potential treatments that satisfied both the first and second criteria; and
providing the second subset to a user.

14. The method of claim 13, wherein the first subset and second subset are stored for future use.

15. The method of claim 13, wherein the treatment repository is RxNav®, a web application program interface that provides a list of ingredients for each active treatment communicated.

16. The method of claim 13, wherein at least one additional criteria is identified and applied to the one or more potential treatments.

17. A system for dynamically managing treatments for one or more conditions, the system comprising:
receiving an indication to provide one or more active treatments associated with one or more conditions for an individual;
retrieving the one or more active treatments associated with the one or more conditions for the individual, wherein the one or more active treatments comprises a unique identifier;
communicating at least one of the one or more active treatments to a treatment repository;
identifying, from the treatment repository, at least one active ingredient of the one or more active treatments communicated;
identifying one or more potential treatments associated with the one or more conditions;
applying a filter comprising the at least one active ingredient to the one or more potential treatments associated with the one or more conditions;
identifying a first subset, wherein the first subset comprises the one or more potential treatments that comprise the at least one active ingredient identified;
identifying a delivery mechanism for the one or more potential treatments;
applying a filter comprising the identified delivery mechanism to the first subset;
identifying a second subset, wherein the second subset comprises the one or more potential treatments that comprise the at least one active ingredient and the identified delivery mechanism;
providing the second subset to a user; and
storing the first and second subsets for future use.

18. The system of claim 17, wherein the delivery mechanism comprises at least one of oral delivery or intravenous delivery.

19. The system of claim 17, wherein the system identifies a third criteria comprising a dosage of the one or more potential treatments.

20. The system of claim 17, wherein the stored first and second subsets are retrieved for use in the management of treatments for the same one or more conditions for a second individual.
